(19) United States
(12) Patent Application Publication
(10) Pub. No.: US 2019/0185818 A1
(43) Pub. Date: Jun. 20, 2019

(54) NOVEL CHICKEN EGG-BASED METASTASIS MODEL FOR CANCER

(71) Applicant: Baylor College of Medicine, Houston, TX (US)

(72) Inventors: Andrew G. Sikora, Houston, TX (US); Ravi Pathak, Houston, TX (US); Rebeca San Martin, Houston, TX (US); David R. Rowley, Houston, TX (US)

(21) Appl. No.: 16/327,291

(22) PCT Filed: Aug. 24, 2017
(86) PCT No.: PCT/US17/48452

§ 371 Date: Feb. 21, 2019

Related U.S. Application Data
(60) Provisional application No. 62/380,449, filed on Aug. 28, 2016.

(51) Int. Cl. C12N 5/09; C12M 3/00; C12N 5/077; G01N 33/50

(52) U.S. Cl. CPC C12N 5/0693; C12M 21/08; C12N 5/0654; G01N 33/5088; G01N 33/5011; C12N 2502/1394; C12N 2533/90; C12N 2502/30; C12N 2502/1352; C12N 2502/1311; G01N 33/5076

(57) ABSTRACT

Embodiments of the present disclosure concern systems, methods, and compositions for both in vitro and in vivo models of metastases, such as bone metastases. In specific embodiments, there is a system comprising a source of bone cells, such as osteoblasts, and a source of cancer cells, wherein the bone cells and cancer cells are configured in a chamber or on a chick chorioallantoic membrane such that interaction between the cells is determined. In specific embodiments, the bone cells are comprised in an organoid comprising both mesenchymal stem cells and osteoblasts (although a naturally derived bone scaffold may be employed), and the cancer cells are comprised in an organoid comprising mesenchymal stem cells and the cancer cells.

This application claims priority to U.S. Provisional Patent Application Ser. No. 62/380,449, filed Aug. 28, 2016, which is incorporated by reference herein in its entirety.

This invention was made with government support under U01CA143055, awarded by National Institutes of Health. The government has certain rights in the invention.

### TECHNICAL FIELD

Embodiments of the present disclosure concern at least the fields of molecular biology, cell biology, drug design, and medicine, including at least cancer medicine.

### BACKGROUND

The stroma plays an important role in the maintenance of tissue homeostasis. Stroma associated with secretory epithelium initiates a wound repair response in the event of a breach in the epithelial layer. This “reactive stroma” response is characterized by the accumulation of myofibroblasts and the remodeling of the extracellular matrix. This response initiates early in prostate cancer, co-evolves with the disease, and is predictive of recurrence.

The present disclosure satisfies a longfelt need in the art to provide effective in vitro and in vivo metastasis models to characterize cancer metastases and their interactions with the microenvironment in a reproducible and accurate manner, such as for bone metastases.

### BRIEF SUMMARY

Embodiments of the disclosure include metastases models and methods of their manufacture and use. In specific embodiments, the metastases model is utilized to study the molecular and biochemical mechanisms involved in metastases, such as bone metastases. Such mechanisms include those that govern the establishment, growth and activity of tumors in the bone, for example. In certain embodiments, a bone metastases model is utilized to study one or more compounds for their efficacy of impeding or preventing bone metastases. Although the primary tumor from which the bone metastases originate may be from any cancer, in specific embodiments it is from prostate, lung, breast, thyroid, renal, myeloma, cervical, head and neck squamous cell carcinomas, or kidney cancer, for example. The disclosure encompasses both in vivo and in vitro models.

In particular embodiments, the disclosure concerns models in which at least one source of cancer cells is exposed to at least one source of cells of a tissue for a tissue metastasis model and/or at least one source of cells capable of differentiating to cells of the tissue (in specific cases the cells are of a cancer's origin or MSCs that elicit a reactive tissue phenotype). Upon exposure of the two types of cells, their interaction may be analyzed in one of a variety of ways, such as for one or more initiating and/or facilitating pathway components and/or for means of manipulating the interaction. In specific embodiments, the interaction is exploited to identify one or more agents that can inhibit at least some aspect of the interaction. Although in many embodiments disclosed herein the source of cells from a tissue being analyzed for a reactive tissue phenotype is from bone, the models and compositions of the disclosure may be applied to any other type of tissue. Although in specific embodiments the source of cells from a tissue being analyzed for a reactive tissue phenotype is a type of bone source, in other embodiments the source is brain (neurosphere organoids); liver (primary hepatic explants or liver organoids); lung (pulmonary organoids/“mini lungs”); or skin (skin organoids derived from primary keratinocytes, as examples.

In particular embodiments, the model is an in vivo model. In such cases, 3-D organoids comprising a mixture of cancer and mesenchymal stem cells are co-implanted with a source of bone and/or a bone substitute (including humanized trabecular bovine bone chips, for example) onto a chick chorioallantoic membrane (CAM), to track the metastatic potential of the cancer cells and/or to test potential drug candidates. In particular embodiments the methods utilize an optimized organoid to bone ratio in the presence of attachment factors and/or extracellular matrix proteins (for example, tenascin C). In some embodiments an organoid is not employed in lieu of seeding a particular cell line and/or explants (for example of any type of cancer) onto the CAM that in specific embodiments may grow in 3D form on the CAM. The cell line may be a cancer cell line, patient-derived stable cell line (for example, derived by ROCK inhibitor or other known methods), patient-derived short-term cell lines, or even small explants derived from a patient or from an existing egg or mouse patient-derived xenograft (PDX) model.

In other embodiments, the model is an in vitro model. In such cases, organoids and/or cell lines and/or explants comprising a mixture of cancer and mesenchymal stem cells are co-cultured with a source of bone and/or a bone substitute (including humanized trabecular bovine bone chips, for example) into a chamber to examine the metastatic potential of the cancer cells and also to test potential drug candidates.

Embodiments of the disclosure include methods of generating osteogenic organoids, including for engraftment onto a CAM. Embodiments also include steps of generating humanized bovine bone chips. In particular embodiments, the disclosure encompasses methods of co-culturing organoids and bone (or a bone source) on a CAM. Embodiments of the disclosure also include methods to image organoids under live circumstances, for example using an in vivo imaging system.

In addition to facilitating the study of metastases in an in vitro and an in vivo system, the models may be used to identify useful agents to treat or prevent metastasis and also to optimize proper dosages of a particular agent, for example. In embodiments wherein one or more potential therapeutic agents are tested in models of the present disclosure, the potential therapeutic agent may be of any kind, including a small molecule, nucleic acid, peptide or polypeptide, antibody, cell-based therapeutic, or a combination thereof.

In one embodiment, there is a bone cancer metastasis model system, comprising: a) a composition comprising at least one source of osteoblasts and/or at least one source of cells capable of differentiating to osteoblasts; b) a composition comprising at least one source of cancer cells; and c) a substrate onto which or into which the compositions in a) and b) are configured. In certain embodiments, the composition in a) comprises: 1) a bone scaffold derived from natural bone; 2) mesenchymal stem cells, osteoblasts, or a mixture thereof; or 3) a combination of 1) and 2), and optionally comprises 4) one or more types of immune cells. In certain embodiments, the composition in 1) comprises bone scaffold and one or more human extracellular matrix proteins.

In at least some cases, the bone scaffold is coated with one or more human extracellular matrix proteins, such as one or more of tenascin C, fibronectin, collagen, laminin, and derivatives thereof. In certain aspects, the bone scaffold is derived from bovine bone. The bone scaffold may be comprised of fragments of at least 200 microns in size and/or comprised of fragments of no more than 500 microns in size. In specific cases the bone scaffold is comprised of fragments of about 0.5 cm³ in size.

In embodiments of a composition that comprises mesenchymal stem cells, osteoblasts, or a mixture thereof, the composition may comprise an organoid comprising a mixture of the mesenchymal stem cells and in situ-differentiated osteoblasts. In specific cases the organoid comprises a mesenchymal stem cell core surrounded by one or more layers of osteoblasts. The mesenchymal stem cells may be prostate-derived mesenchymal stem cells or bone marrow-derived mesenchymal stem cells, as examples.

In cases wherein there is a combination of a bone scaffold and mesenchymal stem cells, osteoblasts, or a mixture thereof, the combination may comprise bone scaffold and at least one layer of osteoblasts on the surface of the scaffold.

In compositions comprising at least one source of cancer cells, composition may comprise cancer cells from at least one prostate, breast, or lung cancer cell line, as examples. The composition may comprise an organoid comprising mesenchymal stem cells and at least one source of cancer cells. In certain cases, the organoid comprises a mesenchymal stem cell core surrounded by one or more layers of the cancer cells. The composition comprising the cancer cells may have mesenchymal stem cells that are bone marrow-derived or organ-derived.

In specific embodiments for the substrate, the substrate comprises a chamber having a non-adherent surface, or the substrate may comprise a chick chorioallantoic membrane (CAM) model. In situations wherein a CAM model is employed, the compositions of the system may be configured within the boundaries of a physical barrier on the CAM, wherein the barrier comprises an aperture allowing exposure of the compositions to the egg. The physical barrier may be ring-shaped, elliptical-shaped, square-shaped, rectangular-shaped, or triangular-shaped.

The some system embodiments, the compositions reside on a protein-based matrix within the boundaries of the physical barrier, and the matrix may be gelatinous, for example being comprised of about 0.1% gelatin. In situations wherein the substrate comprises a chamber having a non-adherent surface, the system is under conditions of 37° C. and/or 5% CO₂.

In one embodiment, there is a kit comprising a system encompassed by the disclosure, wherein the system, compositions of the system, and/or reagents used to generate the compositions are housed in one or more suitable containers.

In an embodiment, there is provided herein a method of using any system encompassed by the disclosure, comprising the steps of generating, providing or obtaining the system; and 1) exposing the system to one or more detection procedures to detect one or more compositions of the system and/or to detect one or more parts of one or more compositions of the system, and/or 2) providing one or more potential therapy agents to the system. In specific embodiments, the one or more detection procedures comprises imaging of one or more compositions of the system and/or one or more parts of one or more compositions of the system. In specific embodiments, the exposing step precedes the step of providing one or more potential therapy agents to the system, although in some cases the step of providing one or more potential therapy agents to the system precedes the exposing step.

In embodiments wherein detection of one or more elements of the system is utilized, the detection procedure images one or more proteins of cells in the system; or one or more nucleic acids of cells in the system. The detection procedure may comprise immunohistochemistry, in situ hybridization, bioluminescence, or a combination thereof.

In cases wherein the system is utilized to screen one or more agents for a therapy agent, the agent comprises an immunotherapy agent, a drug agent, a hormone agent, targeted therapy agent, antibody, aptamer agent, bioactive DNA or RNA agent (e.g. microRNA, shRNA, siRNA), cellular therapeutic, or a combination thereof. In specific cases, when a potential therapy agent is provided to the system, one or more characteristics in the system are determined, such as ablation of migration of cancer cells towards the bone component, decreased colonization of bone, and/or decreased growth in the bone, as examples. In specific cases, when the potential therapy agent ablates migration of cancer cells towards bone cells, decreases colonization of bone, and/or decreases growth in the bone, the potential therapy agent is a bone metastasis therapy agent, and a therapeutically effective amount of the bone metastasis therapy agent is provided to an individual that has cancer or is at risk for having metastasis of cancer.

In one embodiment, there is provided a method of generating a system encompassed by the disclosure, comprising the steps of producing or obtaining a composition comprising at least one source of osteoblasts and/or at least one source of cells capable of differentiating to osteoblasts; and/or producing or obtaining a composition comprising at least one source of cancer cells; or a combination thereof. In some embodiments, when the bone composition comprises bone scaffold, the step of producing the bone composition comprises subjecting the bone scaffold to one or more human extracellular matrix proteins. When the bone composition comprises an organoid comprising a mixture of mesenchymal stem cells and osteoblasts, the step of producing the bone composition may comprise exposing mesenchymal stem cells to sufficient conditions to establish mesenchymal stem cell spheroids that are then exposed to osteogenic media for a sufficient period of time, thereby producing an organoid comprising a mixture of mesenchymal stem cells and osteoblasts. In specific embodiments, a sufficient period of time to establish mesenchymal stem cell spheroids comprises about 24 hours. In certain cases, a sufficient period of time to expose the mesenchymal stem cell spheroids to osteogenic media to produce the organoid is about 7-14 days. In certain cases, the producing step occurs on or in the substrate, and the substrate may be a chamber. In certain aspects, exposing of the mesenchymal stem cells to sufficient conditions to establish mesenchymal stem cell spheroids occurs in a media comprising Dulbecco's modified eagle medium (high glucose), fetal bovine serum, NuSerum™, testosterone, insulin, and one or more antibiotics.

In some cases, an organoid comprising mesenchymal stem cells and cancer cells is provided to a chamber or CAM model either of which comprise 1) the organoid comprising the mixture of mesenchymal stem cells and osteoblasts, or 2) the bone scaffold. The organoid comprising the mesenchymal stem cells and cancer cells may be provided to the chamber within seven days after the organoid comprising the mixture of mesenchymal stem cells and osteoblasts exhibits one or more characteristics of osteogenic induction, such as when the organoid comprising the mixture of mesenchymal stem cells and osteoblasts extends one or more tendrils from the organoid; turns opalescent, white and hard; or both. The organoid comprising the mesenchymal stem cells and cancer cells may be provided to the chamber concomitant with the bone scaffold is provided to the chamber or on the CAM model. In specific embodiments, the bone scaffold is coated with at least one extracellular matrix protein, such as one or more of tenascin C, fibronectin, collagen, laminin, or derivatives thereof.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawing, in which:

FIGS. 1A-1G. MSC Derived Osteogenic Organoids. 1A. Experimental Setup 1B. The osteogenic organoid develops endosteal tendrils that tether it to the culture vessel after seven days of induction. 1C. Immunofluorescence, Osteocalcin 1D. 1H & 1E staining. 1E, 1F, and 1G. Immunohistochemistry for alkaline bone phosphatase, tenascin C and SPARC.

FIG. 2. Illustration of embodiments of the chick corioalantonic membrane (CAM) system utilizing organoids and a bone source.

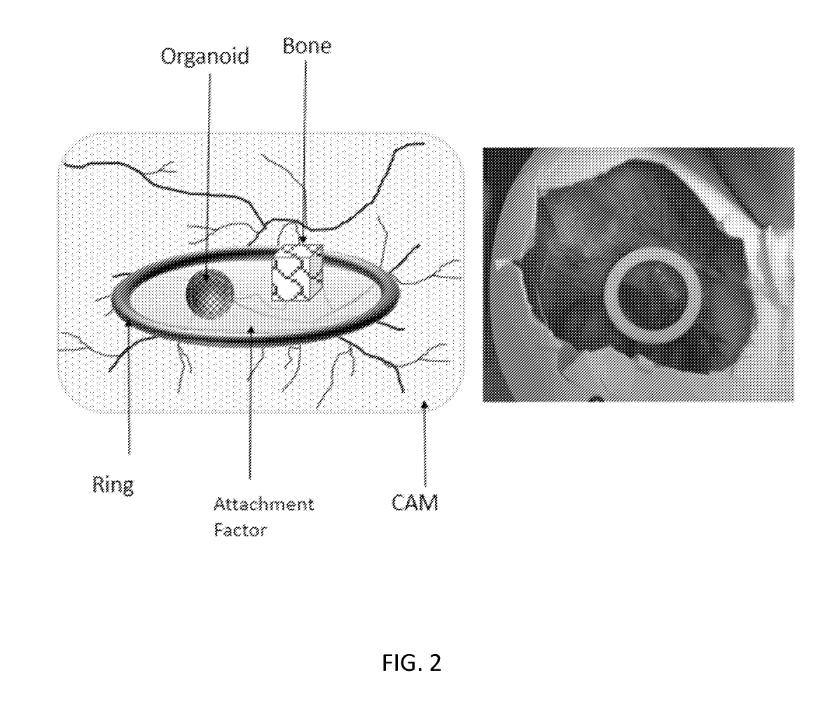

FIG. 3. An example of results from a CAM-Humanized bovine bone integrated experimental system using immunohistochemistry and DNA in situ hybridization.

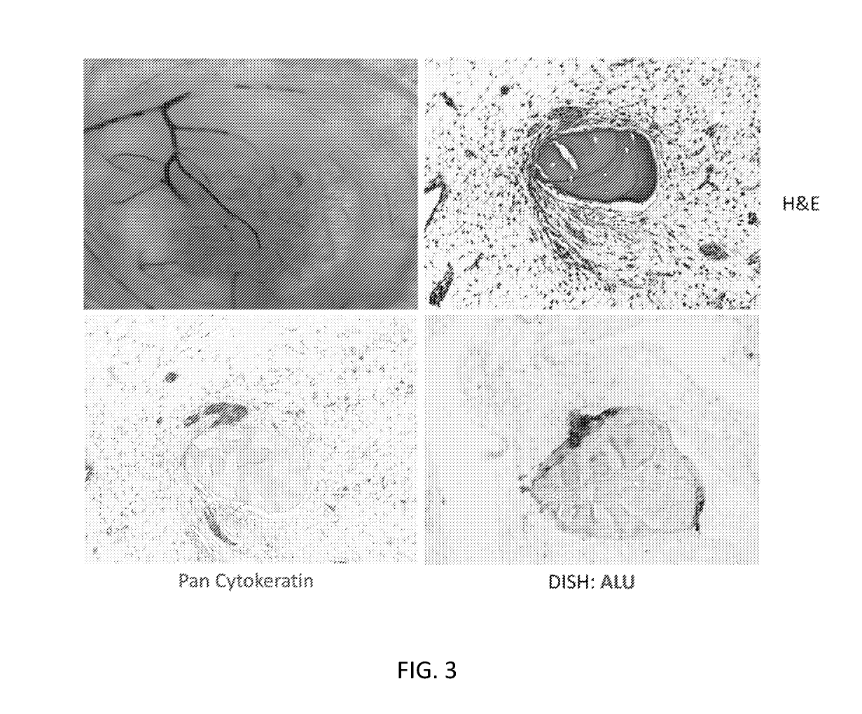

### DETAILED DESCRIPTION

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein.

The term “organoid” as used herein refers to a biological entity, commonly in the shape of a three-dimensional spheroid or sheet, derived from the in vitro co-culture of two or more cell lines that has spontaneously aggregated into a manipulable unit.

### I. General Embodiments

Metastases are a common occurrence in cancer and often cause significant morbidity and mortality. The methods and compositions of the present disclosure enable in vitro and in vivo cancer metastasis modeling, such as for bone as an example. In particular, the present disclosure concerns methods and compositions suitable for characterizing metastases. In particular embodiments, metastases are examined using model(s) that may be utilized for analyzing one or more agents for treating the metastases. As an example, bone metastases, such as from prostate and breast cancer as examples, are examined using model(s) of bone metastasis, and the models may be utilized for analyzing one or more agents for treating bone metastases. In particular, the methods and compositions of the disclosure provide for a better understanding of the molecular mechanisms that govern metastasis of tumors to the bone. While there are a considerable number of cancer models available for scientific studies, few of them can be used to consistently model bone metastases, for example as it occurs in men with prostate cancer. The present disclosure provides a quick, robust and cost effective model to characterize bone metastases in prostate cancer (as an example only) that can be extended for other cancer types.

### I. Embodiments of the System

The present disclosure concerns in vivo and in vitro systems for bone metastases. In both types of systems, there is a composition that provides the bone cells and a composition that provides the cancer cells, both of which are configured on or in a substrate. In certain embodiments, for an in vitro system the components are utilized in a substrate, and in other embodiments for an in vivo system the components are utilized on a substrate.

In some embodiments, there is a bone cancer metastasis model system, comprising a composition comprising at least one source of bone cells, such as osteoblasts, and/or at least one source of cells capable of differentiating to bone cells, such as osteoblasts. The system also comprises a composition comprising at least one source of cancer cells and, optionally (or not) a substrate onto which or into which the bone and cancer compositions are configured.

### A. Embodiments of Sources of Cells from a Tissue being Analyzed

In methods and compositions of the disclosure that model tissue or cell metastasis, cells of a tissue and/or at least one source of cells capable of differentiating to cells of the tissue are analyzed. In specific cases, the cells are long term-culture stromal cells of a cancer's origin or MSCs eliciting a reactive tissue phenotype, a component of the metastatic process. One may employ a source of the cells that includes organoids, explants, and/or cell lines. In methods and compositions of the disclosure that model bone metastasis, one may employ a source of bone cells, including osteoblasts, and/or fragments of bone, and/or similar materials. In specific embodiments, the source of bone cells includes at least one type of bone matrix, whether the bone matrix comes from the same source of the bone cells themselves (for example, in natural bone) or whether it is provided exogenously to a plurality of bone cells. The matrix may be of natural materials or non-natural materials, so long as they mimic natural bone materials (as an example, a three-dimensional, solid, hydroxyapatite matrix). Natural bone or synthetic bone substitutes or natural bone substitutes may be used.

In some cases, bone chips are utilized because they are a naturally occurring three dimensional, hydroxyapatite matrix where metastatic cells naturally grow. A bone matrix of porous bone from a human or non-human animal (including bovine bone) may be utilized, in some cases. Demineralized bone matrix may be used, in some cases. The compositions may include mineralized collagen matrix or cortical cancellous chips, for example. In specific embodiments, Nukbone® is utilized. In any such case, the bone source may comprise one or more extracellular matrix proteins.

In particular embodiments, the source of bone cells comprises cells capable of differentiating into bone cells, including differentiating into osteoblasts. Such cells may be of any kind, but in particular embodiments the cells are stem cells or progenitor cells. In such cases, the cells capable of differentiating into bone cells are cultured under conditions suitable for differentiating into bone cells such as osteoblasts. In cases wherein stem cells are utilized, the stem cells may be of any kind, including mesenchymal stem cells. The stem cells are adult stem cells, in specific embodiments. In specific embodiments, the mesenchymal stem cells are organ-derived stem cells, including from the prostate. The mesenchymal stem cells may be used from the prostate, placenta, adipose tissue, lung, bone marrow and blood, Wharton's jelly from the umbilical cord, and teeth, for example. In some cases, the cells are from an individual that is to be treated, including an individual with bone cancer metastasis, although in other cases the cells are from an individual different from the one to be treated. In particular embodiments, the stem cells or progenitor cells are differentiated such that the resultant cells exhibit certain markers, such as one or more of osteocalcin, alkaline phosphatase, SPARC, tenascin C, and so forth. The presence of the markers may be assayed using qPCR, immunohistochemistry, or both, for example. In some cases, the system procedures are sufficiently established that it is not necessary to assay for the presence of one or more markers.

In particular embodiments, the composition comprising the bone cells (including osteoblasts) with or without mesenchymal stem cells is configured in an organoid. In specific embodiments, the outside layer(s) of the osteogenic organoid is comprised of osteoblasts and the core comprises undifferentiated mesenchymal stem cells.

In specific embodiments, the composition comprising the source of bone cells includes one or more extracellular matrix proteins, and in specific embodiments the extracellular matrix proteins are human proteins. The proteins may be coated onto bone fragments, in some cases. Tenascin C may be coated by immersion onto bone scaffold. In certain embodiments, other extracellular matrix proteins may be coated by immersion, given the porous nature of the scaffold. In specific embodiments the extracellular matrix protein is one or more of tenascin C, fibronectin, collagen, laminin, or derivatives thereof, for example.

In specific embodiments, a bone scaffold having fragments of a certain size are utilized. In specific cases the bone scaffold is comprised of fragments of at least 200 microns in size and may be comprised of fragments of no more than 500 microns in size. In certain cases, the bone scaffold is comprised of fragments of about 0.5 cm³ in size.

### B. Source of Cancer Cells

The system includes a composition that comprises cancer cells. The cancer cells may be from a cell line, such as a commercially-obtained or research institution-obtained cell line, or they may be from an individual with cancer, for example. In cases wherein cell lines are utilized, there may be used mixtures of different cell lines of the same type of cancer. The cancer cells may be of any type of cancer, including prostate, breast, lung, or kidney, for example. The cell line may be a cancer cell line, patient-derived stable cell line, a patient-derived short-term cell line, and/or may not be a cell line but is an explant, such as a PDX explant derived from a patient or another model, such as another CAM model, for example an egg or mouse PDX model.

In particular embodiments, the composition that comprises the cancer cells are also comprised of stem cells or progenitor cells, such as mesenchymal stem cells. The cells may be fibroblasts. The stem cells may be organ-derived, including prostate-derived, and they may be bone marrow-derived. The stem cells, or tumor initiating cells, may be derived from circulating tumor cells. In such cases, the stem cells and cancer cells may be configured in an organoid, and the organoid may comprise a mesenchymal stem cell core surrounded by one or more layers of the cancer cells. One of skill in the art is aware of procedures to produce cancer cell-comprising organoids (Kim et al., 2014).

### C. Substrate

In particular embodiments, the system utilizes a substrate for the bone source composition and cancer cell composition to reside in or on. The substrate may be of any kind, but in particular cases the substrate is part of an in vitro model or an in vivo model. In some cases, in cases where organoids are used, the organoid(s) may be generated on or in the substrate prior to use as a metastasis model in the system.

### 1. In Vitro Model

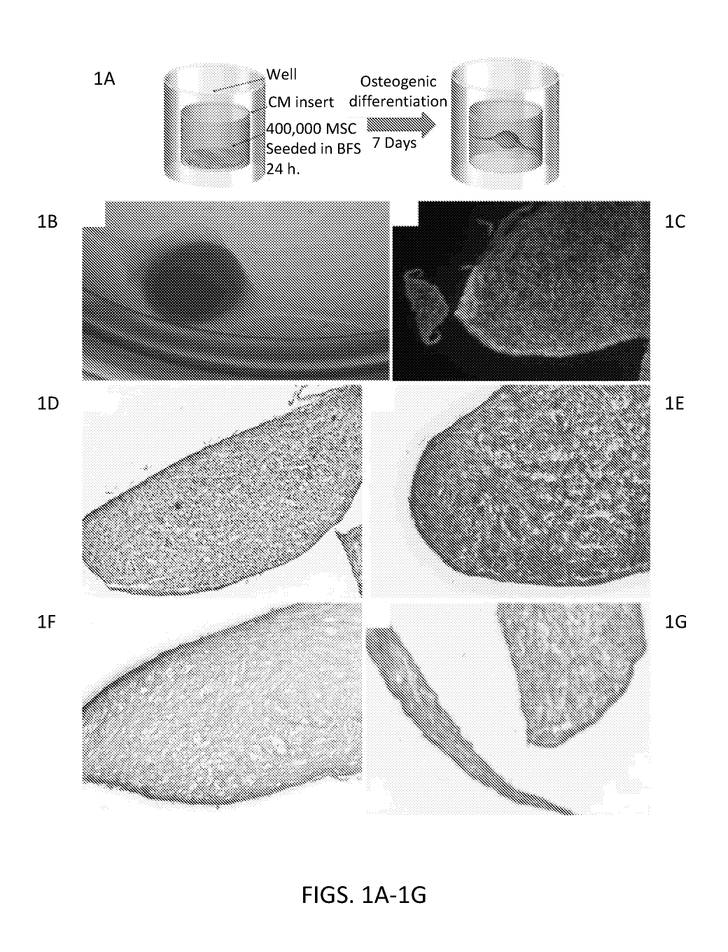

In particular embodiments, the substrate is part of an in vitro model, and in such cases the substrate comprises a non-adherent surface so that the cells in the system will not adhere to the surface but instead to each other. In particular embodiments, the in vitro model utilizes wells or chambers in which the organoids or bone scaffold are placed. FIG. 1 illustrates an embodiment of a particular cell culture insert that may be utilized. As an example, Millipore® Millicell® cell culture inserts may be utilized.

In specific embodiments, the substrate is a plastic vessel with a non-adherent but porous membrane at the bottom to allow flow of nutrients from the outside media chamber. The non-adherent nature of the membrane compels the cells to attach to each other and form a ball in the center. Unlike other organoid systems, the organoids of the present disclosure do not require a matrix substrate (such as Matrigel®) to form.

### 2. In Vivo Model

In particular embodiments an in vivo model is used in systems and methods of the disclosure. In specific embodiments, the in vivo model utilizes a chick chorioallantoic membrane (CAM) model that utilizes the vascular membrane as a source of nutrients. The CAM model may be generated by methods described in U.S. Provisional Patent Application Ser. No. 62/251,404 and PCT Application Serial No. PCT/US2016/060664, which are incorporated by reference herein in their entirety, in addition to methods known in the art. Generally, fertilized chicken eggs (for example, 6-, 7-, 8-, 9-, or 10-day old) are incubated in a humidified 37° C. chamber. Under sterile conditions, the eggshell surface is cleaned, a small hole is introduced (such as with a 19-G needle (egg hole punch)) in the air sack, and a window is created.

In particular embodiments, the bone source composition and the cancer cell composition (both of which may be organoid(s)) are placed on the membrane within the boundaries of a physical barrier on the CAM, wherein the barrier comprises an aperture allowing exposure of the compositions to the egg. The barrier may be of any suitable shape, including a ring, ellipse, square, rectangle, triangle, and so forth. The barrier is of a sufficient size to allow the presence of multiple organoids, in some cases. The barrier may be made of material that is biologically inert, such as of at least one silicon-based organic polymer, including polysiloxanes or fluoropolymers; one example is Teflon®.

In specific cases, the compositions reside on a protein-based matrix within the boundaries of the physical barrier. In specific embodiments the matrix is gelatinous, such as being comprised of about 0.1% gelatin. A range of percentage of gelatin may be employed, such as 0.01% gelatin to 1% gelatin. In specific embodiments, protein-based gel matrices include attachment factor, gelatin, Matrigel®, Geltrex®, and so forth. The protein-based matrix prevents the bone from sinking and potentially puncturing the CAM, and it also provides a sticky area for the organoid to reside and remain in position until the vasculature migrates in.

In particular embodiments the CAM model is maintained under suitable conditions, such as being about 37° C. and moist.

### II. Methods of Making the System

Methods of making the system of the disclosure are encompassed herein. The different compositions of the system may be generated at different times or at the same time. The system composition(s) may be generated or obtained prior to use, although in at least some cases the nature of the cells prohibits development of the models far in advance of their use. In certain embodiments, the system or parts thereof is generated just prior to use, and in some cases the system is generated from cancer cells from an individual in need of cancer therapy. In specific embodiments, an individual in need of cancer therapy may be at risk for metastases or is known to have metastases. The system may be used for individuals with cancer to determine a proper dosage, to tailor a personalized therapy to the individual, and so on.

### A. Generation of Osteogenic Organoids

In some embodiments, a source of bone cells, such as osteoblasts, is present in the system in the form of an organoid comprising a mixture of cells. The organoid may be generated prior to placement in or on the substrate, or the organoid may be generated in or on the substrate. In specific embodiments, the mixture comprises stem cells (such as mesenchymal) and osteoblasts, and the osteoblasts may be differentiated from the stem cells (including mesenchymal) under appropriate conditions. Osteoblasts are utilized in particular embodiments because that is the cell to which the metastatic cells will hone upon arrival to the trabecular bone.

The mesenchymal stem cells may be organ-derived, including prostate-derived, for example, or they may be bone marrow-derived. The stem cells may be obtained commercially or from an individual in need of treatment.

In particular embodiments, the osteogenic organoid is grown in a particular media, such as an osteogenic media, and in at least certain cases the organoid is not grown in the same media as the mesenchymal stem cells that are cultured prior to development of the organoid.

In general embodiments, mesenchymal stem cells are grown in a standard media for their growth until reaching a certain confluency (for example, 80% confluency. The cells are trypsinized and washed in another media (for example, BFS media), followed by centrifugation. Cell concentration is adjusted to a desired concentration (for example, 400,000 cells per 300 microliters). In some embodiments, 200,000 to 800,000 cells are utilized. Certain aliquots (for example, 300 microliter aliquots) are then seeded into a culture chamber (for example, CM membrane inserts (Millipore)) and organoids are allowed to form at least for about 24 hours. After organoids are formed, the media is switched to osteo-inductive media (for example, from R&D Systems, Inc.; Minneapolis, Minn.) for 7-24 days.

In specific embodiments, once the osteogenic organoid or bone scaffold extends “tendrils” out of the organoid's or scaffold's core, the cells that populate the surface of the organoid and tendrils are osteoblasts and are ready for use in the system.

As disclosed herein, organoids that are not bone organoids may be employed to analyze metastasis of other tissues, such as neurosphere organoids for the brain, primary hepatic explants or liver organoids for the liver, pulmonary organoids or “mini lungs” for the lung, and skin organoids for the skin, for examples.

### B. Generation of Cancer Organoids

The system utilizes an organoid comprised of stem cells, including mesenchymal stem cells, and a particular type of cancer cells, such as from a cell line or from an individual in need of therapy. The generation of such cancer organoids is known in the art (Kim et al., 2014).

For generation of the cancer organoid, the organoid may be cultured in a certain media. Different cell lines require different base media when cultured in vitro, and the skilled artisan recognizes how to determine the appropriate media, for example, as directed by the cancer cell depository institution. For example, prostate cancer cell lines VCaP and PC3 require the base formula DMEM:F12 1:1 Ham (Gibco, ThermoFisher Scientific, Waltham, Mass.), while LNCaP cells require the base formula RPMI (Gibco).

### C. Co-Culture in or on the Substrate

In particular embodiments, free floating cancer cells are co-cultured with the osteogenic organoid or bone scaffold (for example, within about 7 days after osteogenic induction started for the osteogenic organoid embodiment) in or on the substrate.

In cases wherein the source of bone cells (osteoblasts) is the osteogenic organoid, the osteogenic organoid may be generated within the substrate (chamber), and a cancer cells are then added to the chamber. In examples wherein the substrate is the CAM in vivo model, the osteogenic organoid may be generated in a chamber or other environment and then placed on the CAM along with the cancer organoid.

In cases wherein the source of bone cells is bone scaffold, the scaffold may be coated with one or more extracellular matrix proteins and then added to a chamber with cancer cells in suspension or to a CAM model that already has a cancer organoid, or the cancer organoid may be added to the substrate following the bone scaffold.

In specific embodiments, after osteogenic induction the osteoinductive media is removed from the chamber and the cancer cells of choice are added in an aliquot of cancer cell-specific media. Typically, co culture experiments may be terminated by fixation after 48 hours.

Once the bone source (osteogenic organoid or bone scaffold) and the source of cancer cells are placed or generated in the system, the cancer cells migrate to the bone, and this may or may not be observed prior to exposing the system to drug candidate(s) for example. However, the occurrence may be assayed by immunohystochemical and/or expression profile analysis when desired.

In cases wherein the system is utilized for testing of drug or other candidate therapeutic, a suitable amount of the candidate may be provided to the system, and the system is observed for one or more outcomes, such as ablation of migration of cancer cells towards the bone component, decreased colonization of bone, and/or decrease growth in the bone (with bone merely as an example).

### III. Methods of Using the System

The system of the disclosure may be utilized in a variety of ways. In particular embodiments, the system provides information about one or more mechanisms related to metastases of any kind, including bone metastases, and this information may identify particular drug targets, for example. The information from the system may be obtained by one or more detection methods, and such detection methods may utilize direct or indirect imaging, for example. The detection step(s) may detect cells, part or all of the organoid, particular cells within the organoid, and/or subcellular component(s) of cells within the organoid. Certain subcellular components include one or more types of proteins and/or nucleic acids, for example. The detection methods include immunohistochemistry, in situ hybridization, bioluminescence, polymerase chain reaction, or a combination thereof, for example.

In some embodiments, the system is used to identify useful therapy agents. For example, potential drug candidate(s) may be exposed to the system to determine if they are able to provide a useful outcome in the context of the metastasis model. With bone as merely an example, certain characteristics to consider for an output for the model include ablation of migration of cancer cells towards the bone component, decreased colonization of bone, and/or decrease growth in the bone. Particular types of drug candidates for testing include small molecules, nucleic acids, and/or proteins, including antibodies, for example.

In particular embodiments, a plurality of in vitro and/or in vivo models of the present disclosure are established, and then multiple drug candidates are examined in the models, including concomitantly. In some cases part or all of a library of candidates are examined in the models. The output of the model being used for such drug testing may be qualitative or quantitative. For example, particular doses may be examined with the same drug or drug candidate using models of the disclosure.

### EXAMPLES

The following examples are included to demonstrate preferred embodiments of the disclosure. It should be appreciated by those of skill in the art that the techniques disclosed in the examples that follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1

### Tenascin C as an Effector of Prostate Cancer Derived Bone Metastasis

To assess whether bone exhibits a reactive tissue phenotype in the context of metastasis, human prostate-derived bone metastasis tissue arrays were evaluated using immunohistochemistry and spectral deconvolution. This work identified tenascin C expression in trabeculae-associated metastatic sites. Because tenascin C expression in adult differentiated bone is restricted to regions of repair, the inventors considered this cancer associated phenotype as a reactive endosteum. In order to evaluate the mechanisms involved, they developed an in vitro 3D osteogenic organoid, using human mesenchymal stem cells induced to osteoblastic differentiation. Co-culture with the metastatic prostate cancer cell line VCaP showed preferential binding at sites high in tenascin C deposition. Metastatic cells also adhere to osteo-mimetic surfaces coated with tenascin C in vitro, showing an accelerated growth rate and forming 3D colonies. It was determined that a9b1 integrin is an important mediator of prostate cancer cell adhesion to tenascin C. Finally, preliminary results from xenograft experiments on the chorioallantoic membrane of the chicken egg have shown that metastatic cells preferentially migrate and colonize bone trabecular scaffolds coated with tenascin C.

These studies characterize a reactive endosteum phenotype at sites of metastatic prostate cancer and suggest that elevated tenascin-C mediates adhesion and favors growth of cancer cells and will provide insights from which to develop novel therapeutic approaches to treat metastatic disease.

### Example 2

### In Vitro MSC-Derived 3D Endosteal Organoid Model

Mesenchymal Stem Cellls (Lonza), growing in T75 cell culture flasks (Gibco) were collected and washed twice with BFS media (10 ml) (DMEM; high glucose (GIBCO) supplemented with 5% (vol/vol) FBS (HyClone), 5% (vol/vol) NuSerum (Collaborative Research), 0.5 μg/mL testosterone, 5 μg/mL insulin, 100 units/mL penicillin, and 100 μg/mL streptomycin (Sigma)]) by centrifugation (400 rpm, 3 min). The cell pellet was re-suspended in 2 ml of BFS media and cell count determined by trypan blue exclusion. BFS was added to the cell suspension to reach a concentration of 400,000 cells/300 μl. Cell culture inserts (Millipore. Millicell-CM 12 mm) were prepared as suggested by the manufacturer; 600 μl of BFS media was added to each well of a 24 well plate and inserts were transferred into the media under aseptic conditions. Once the membranes were moist, each chamber was seeded with 300 μl of the cell suspension.

After overnight incubation, once MSC spheroids were formed, the BFS media in both the inner and outer chambers was carefully removed and substituted with complete osteogenic media (R&D CCM007 supplemented with CCM008). Osteogenic organoids were cultured for 7, 14, and 21 days, with media changes every two days.

At seven days of osteogenic induction in non-adherent conditions, mesenchymal cells aggregates differentiate into hard, white, opalescent organoids that are free floating but tethered to the sides of the cell culture insert by distinct, fibrous and flexible tendrils (FIG. 1B). Histological analysis of the organoids revealed that the mass of cells is surrounded by a distinct, flat and compact, layer of cells that resembles the endosteal layer associated with trabecular bone (FIG. 1D). Positive differentiation of these cells into the osteoblastic phenotype was confirmed by immunostaining of osteocalcin, alkaline phosphatase, SPARC and interestingly, Tenascin C. Small foci of matrix deposition were also observed in all induced organoids.

### Example 3

### The Cam-Humanized Bovine Bone Integrated Experimental System

NukBone® (Biocriss; Mexico City, MX), is a trabecular bone scaffold of bovine origin, which has been shown to foster MSC differentiation into the osteoblastic phenotype, and is used as an implant to aid bone repair in human patients (Pin̆a-Barba, 2006; Rodriguez-Fuentes et al., 2013). For the studies, NukBone® chips (0.5 cm) were coated with human, full length, tenascin C (Millipore) at 100 mg/ml for one week or BSA as control.

This system uses the chorioallantoic Membrane (CAM) of the chicken egg as a host for a xenograft composed of the “humanized” NukBone® in combination with an organoid consisting of a mixture of VCaP cells (prostate cancer metastatic cell line) and the prostate-derived mesenchymal stem cell 19I (Kim et al., 2014). Briefly, 100 μl of attachment factor (Gibco) is allowed to set as a membrane within the confines of a neoprene ring that lies on top of the exposed CAM. Once the surface turns opalescent, the humanized trabecular bone chip is placed inside the ring, followed by the prostate cell line-derived organoid. The egg is placed in a humidity-controlled incubator at 37° C. for six days.

Initial results from this CAM-Humanized Bovine Bone integrated experimental system (CHuBBies) has shown that trabecular bone coated with full length tenascin C is colonized with epithelial cells that migrate out of the VCaP-19I organoid, which has been confirmed via immunohistochemistry for specific markers (AR, cytokeratin), and DNA in situ hybridization for the ALU sequence to confirm human origin of the epithelium.

### REFERENCES

All patents and publications mentioned in the specification are indicative of the level of those skilled in the art to which the invention pertains. All patents and publications are herein incorporated by reference to the same extent as if each individual publication was specifically and individually indicated to be incorporated by reference

Kim, W., Barron, D. A., San Martin, R., Chan, K. S., Tran, L. L., Yang, F., . . . Rowley, D. R. (2014). RUNX1 is essential for mesenchymal stem cell proliferation and myofibroblast differentiation. Proc Natl Acad Sci USA, 111(46), 16389-16394. doi:10.1073/pnas.1407097111

Pina-Barba, M. C. (2006). Cracterizacion del hueso bovino anorganico: Nukbone. [Characterization of anorganic bovine bone: Nukbone]. Acta Ortopedica Mexicana, 20 (4).

Rodriguez-Fuentes, N., Rodriguez-Hernandez, A. G., Enriquez-Jimenez, J., Alcantara-Quintana, L. E., Fuentes-Mera, L., Pina-Barba, M. C., . . . Ambrosio, J. R. (2013). Nukbone® promotes proliferation and osteoblastic differentiation of mesenchymal stem cells from human amniotic membrane. Biochem Biophys Res Commun, 434(3), 676-680. doi:10.1016/j.bbrc.2013.04.007

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

1. A tissue cancer metastasis model system, comprising:
a) a composition comprising at least one source of cells of the tissue and/or at least one source of cells capable of differentiating to cells of the tissue;
b) a composition comprising at least one source of cancer cells; and
c) a substrate onto which or into which the compositions in a) and b) are configured.

2. The system of claim 1, wherein the tissue is bone and the cells of the tissue are osteoblasts.

3. The system of claim 2, wherein the composition in a) comprises:
1) a bone scaffold derived from natural bone;
2) mesenchymal stem cells, osteoblasts, or a mixture thereof; or
3) a combination of 1) and 2), and optionally comprises
4) one or more types of immune cells.

4. The system of claim 3, wherein the composition in 1) comprises bone scaffold and one or more human extracellular matrix proteins.

5. The system of claim 4, wherein the bone scaffold is coated with one or more human extracellular matrix proteins.

6. The system of claim 4, wherein the extracellular matrix protein is tenascin C, fibronectin, collagen, laminin, or derivatives thereof.

7. The system of claim 3, wherein the bone scaffold is derived from bovine bone.

8. The system of claim 3, wherein the bone scaffold is comprised of fragments of at least 200 microns in size.

9. The system of claim 3, wherein the bone scaffold is comprised of fragments of no more than 500 microns in size.

10. The system of claim 3, wherein the bone scaffold is comprised of fragments of about 0.5 cm³ in size.

11. The system of claim 3, wherein the composition in 2) comprises an organoid comprising a mixture of the mesenchymal stem cells and in situ-differentiated osteoblasts.

12. The system of claim 11, wherein the organoid comprises a mesenchymal stem cell core surrounded by one or more layers of osteoblasts.

13. The system of claim 3, wherein the mesenchymal stem cells are prostate-derived mesenchymal stem cells or bone marrow-derived mesenchymal stem cells.

14. The system of claim 3, wherein the combination in 3) comprises bone scaffold and at least one layer of osteoblasts on the surface of the scaffold.

15. The system of claim 3, wherein the composition of b) comprises cancer cells from at least one prostate, breast, or lung cancer cell line.

16. The system of claim 1, wherein the composition in b) comprises an organoid comprising mesenchymal stem cells and the at least one source of cancer cells.

17. The system of claim 16, wherein the organoid comprises a mesenchymal stem cell core surrounded by one or more layers of the cancer cells.

18. The system of claim 16, wherein the mesenchymal stem cells are bone marrow-derived mesenchymal stem cells or organ-derived mesenchymal stem cells.

19. The system of claim 1, wherein the substrate comprises a chamber having a non-adherent surface.

20. The system of claim 1, wherein the substrate is a chick chorioallantoic membrane (CAM) model.

21. The system of claim 20, wherein the compositions of a) and b) are configured within the boundaries of a physical barrier on the CAM, wherein the barrier comprises an aperture allowing exposure of the compositions to the egg.

22. The system of claim 21, wherein the physical barrier is ring-shaped, elliptical-shaped, square-shaped, rectangular-shaped, triangular-shaped, or amorphously shaped.

23. The system of claim 21, wherein the compositions of a) or b) reside on a protein-based matrix within the boundaries of the physical barrier.

24. The system of claim 23, wherein the matrix is gelatinous.

25. The system of claim 23, wherein the matrix is comprised of 0.1% gelatin.

26. The system of claim 1, wherein when the substrate comprises a chamber having a non-adherent surface, the system is under conditions of 37° C. and/or 5% CO₂.

27. A kit comprising the system of claim 1, wherein the system, compositions of the system, and/or reagents used to generate the compositions are housed in one or more suitable containers.

28. A method of using the system of claim 1, comprising the steps of generating, providing or obtaining the system; and
1) exposing the system to one or more detection procedures to detect one or more compositions of the system and/or to detect one or more parts of one or more compositions of the system, and/or
2) providing one or more potential therapy agents to the system.

29. The method of claim 28, wherein the one or more detection procedures comprises imaging of one or more compositions of the system and/or one or more parts of one or more compositions of the system.

30. The method of claim 28, wherein the exposing step precedes the step of providing one or more potential therapy agents to the system.

31. The method of claim 28, wherein the step of providing one or more potential therapy agents to the system precedes the exposing step.

32. The method of claim 29, wherein the detection procedure images one or more proteins of cells in the system.

33. The method of claim 29, wherein the detection procedure images one or more nucleic acids of cells in the system.

34. The method of claim 29, wherein the detection procedure comprises immunohistochemistry, in situ hybridization, bioluminescence, or a combination thereof.

35. The method of claim 28, wherein the agent comprises an immunotherapy agent, a drug agent, a hormone agent, or a combination thereof.

36. The method of claim 28, wherein when the potential therapy agent is provided to the system, one or more characteristics in the system are determined.

37. The method of claim 36, wherein the one or more characteristics comprise one or more of the following:
ablation of migration of cancer cells towards the bone component,
decreased colonization of bone, and
decrease growth in the bone.

38. The method of claim 37, wherein when the potential therapy agent ablates migration of cancer cells towards bone cells, decreases colonization of bone, and/or decreases growth in the bone, the potential therapy agent is a bone metastasis therapy agent.

39. The method of claim 38, comprising the step of delivering a therapeutically effective amount of the bone metastasis therapy agent to an individual that has cancer.

40. A method of generating the system of claim 1, comprising the steps of:
producing or obtaining the composition of a);
producing or obtaining the composition of b); or
a combination thereof.

41. The method of claim 40, wherein when the composition of a) comprises bone scaffold, the step of producing the composition of a) comprises subjecting the bone scaffold to one or more human extracellular matrix proteins.

42. The method of claim 40, wherein when the composition of a) comprises an organoid comprising a mixture of mesenchymal stem cells and osteoblasts, the step of producing the composition of a) comprises exposing mesenchymal stem cells to sufficient conditions to establish mesenchymal stem cell spheroids that are then exposed to osteogenic media for a sufficient period of time, thereby producing an organoid comprising a mixture of mesenchymal stem cells and osteoblasts.

43. The method of claim 42, wherein the sufficient period of time to establish mesenchymal stem cell spheroids comprises about 24 hours.

44. The method of claim 42, wherein the sufficient period of time to expose the mesenchymal stem cell spheroids to osteogenic media to produce the organoid is about 7-14 days.

45. The method of claim 42, wherein the producing step occurs on or in the substrate.

46. The method of claim 45, wherein the substrate is a chamber.

47. The method of claim 42, wherein the exposing of the mesenchymal stem cells to sufficient conditions to establish mesenchymal stem cell spheroids occurs in a media comprising Dulbecco's modified eagle medium (high glucose), fetal bovine serum, NuSerum™, testosterone, insulin, and one or more antibiotics.

48. The method of claim 42, wherein an organoid comprising mesenchymal stem cells and cancer cells is provided to a chamber or CAM model either of which comprise 1) the organoid comprising the mixture of mesenchymal stem cells and osteoblasts, or 2) the bone scaffold.

49. The method of claim 48, wherein the organoid comprising the mesenchymal stem cells and cancer cells is provided to the chamber within seven days after the organoid comprising the mixture of mesenchymal stem cells and osteoblasts exhibits one or more characteristics of osteogenic induction.

50. The method of claim 49, wherein a characteristic of osteogenic induction is when the organoid comprising the mixture of mesenchymal stem cells and osteoblasts extends one or more tendrils from the organoid; turns opalescent, white and hard; or both.

51. The method of claim 48, wherein the organoid comprising the mesenchymal stem cells and cancer cells is provided to the chamber concomitant with the bone scaffold is provided to the chamber or on the CAM model.

52. The method of claim 51, wherein the bone scaffold is coated with at least one extracellular matrix protein.

53. The method of claim 52, wherein the extracellular matrix protein is tenascin C, fibronectin, collagen, laminin, or derivatives thereof.
